(19) United States
(12) Patent Application Publication
(10) Pub. No.: US 2012/0323181 A1
(43) Pub. Date: Dec. 20, 2012

(54) Intravenous Catheter Introducer with Needle Retraction Controlled by Catheter Hub Seal

(76) Inventors: Thomas J. Shaw, Frisco, TX (US); Ni Zhu, Plano, TX (US)

(21) Appl. No.: 13/494,646

(22) Filed: Jun. 12, 2012

Related U.S. Application Data
(60) Provisional application No. 61/498,230, filed on Jun. 17, 2011.

(51) Int. Cl. A61B 17/34; A61M 25/01

(52) U.S. Cl. 604/164.12; 604/164.01; 604/167.01

(57) ABSTRACT

A passive intravenous (“IV”) catheter introducer having a spring-biased needle retraction system that is triggered when the needle is manually withdrawn through a catheter hub seal. The catheter hub seal constricts to block the rearward flow of bodily fluid and wipe potentially infectious fluids off the outer surface of the needle as the needle is manually withdrawn through the catheter sheath and seal following insertion of the catheter into a patient's vein. No additional steps are needed to activate the needle retraction mechanism beyond manual removal of the needle from the catheter hub seal.

### BACKGROUND OF THE INVENTION

1. Field of the Invention

This invention relates to an intravenous (“IV”) catheter introducer having a spring-biased needle retraction system that is triggered when the needle is manually withdrawn through a polymeric seal disposed inside the catheter hub.

2. Description of Related Art

IV catheter introducers are well known and typically utilize a needle that is inserted through a flexible polymeric sheath to introduce the forwardly extending tip of the sheath into a patient's vein. Such catheter introducers frequently comprise a catheter hub section and a coaxially aligned handle or plunger section. The hub section of some conventional catheter introducers also comprises a laterally disposed fluid port that allows fluid to be introduced into or withdrawn from a patient through a fluid flow path that is established when the needle is withdrawn out of the catheter sheath after the forwardly extending tip of the needle and catheter have been inserted into a patient's vein.

Some conventional IV catheter introducers require manual withdrawal of the needle to a point where the needle tip is no longer disposed inside the hub. Other conventional IV catheter introducers comprise a biasing means such as a needle retraction spring that can be triggered or released to withdraw the needle into the barrel from a position where the needle tip is still disposed inside a patient's vein at the time of triggering. Once a needle has penetrated a patient's skin and vein, clinical practice procedures require that it never be reinserted into a catheter to prevent inadvertent shearing or puncture of the catheter, catheter fragmentation, or catheter embolism.

As a general matter, it is safer for both patients and other healthcare personnel if the needle tip can be automatically retracted inside the barrel or handle following use, particularly if the needle tip carries blood-borne pathogens. Alternatively, where an attempt is made to recap the needle or to place a device having an exposed needle tip directly into a sharps container, accidental needle sticks and the unintended spread of infections are more likely to occur.

### SUMMARY OF THE INVENTION

An IV catheter introducer with an automatically retractable needle and a cooperating catheter hub seal is disclosed. The catheter hub seal cooperates with the tapered inside wall of the hub to maintain the needle and needle holder in a desired position relative to the catheter hub and sheath and to resist the rearwardly directed biasing force of a retraction spring during prior to retraction; wipes potentially infectious fluids off the outer surface of the needle as the needle is manually withdrawn through the catheter sheath and seal following insertion of the catheter into a patient's vein; and then desirably constricts after the needle tip is manually withdrawn rearwardly through the seal to block fluid flow rearwardly out of the hub as fluid is injected into or withdrawn through a lateral access port.

### BRIEF DESCRIPTION OF THE DRAWINGS

The apparatus of the invention is further described and explained in relation to the following drawings wherein:

FIG. 1 is a front elevation view of a preferred embodiment of an IV catheter of the invention;

FIG. 2 is an enlarged cross-sectional plan view taken along line 2-2 of FIG. 1;

FIG. 3 is a cross-sectional elevation view through the longitudinal axis of IV catheter of FIG. 1; and

FIG. 4 is a cross-sectional view through the barrel portion of the IV catheter of FIG. 3, wherein the retraction spring is relaxed and the needle holder and needle have been propelled rearwardly into a retraction cavity inside the barrel.

### DESCRIPTION OF THE PREFERRED EMBODIMENT

The IV catheter introducer disclosed herein combines automated needle retraction with a catheter hub seal that is already in place, thereby preventing bodily fluids from flowing rearwardly out of the catheter hub once the needle is withdrawn past the seal. This is a wholly passive medical catheter introducer in that no additional steps are needed to activate the needle retraction mechanism beyond manual removal of the needle from the catheter hub seal. Incremental reinsertion of the needle into the catheter sheath is prevented by rearward, unidirectional movement of the needle.

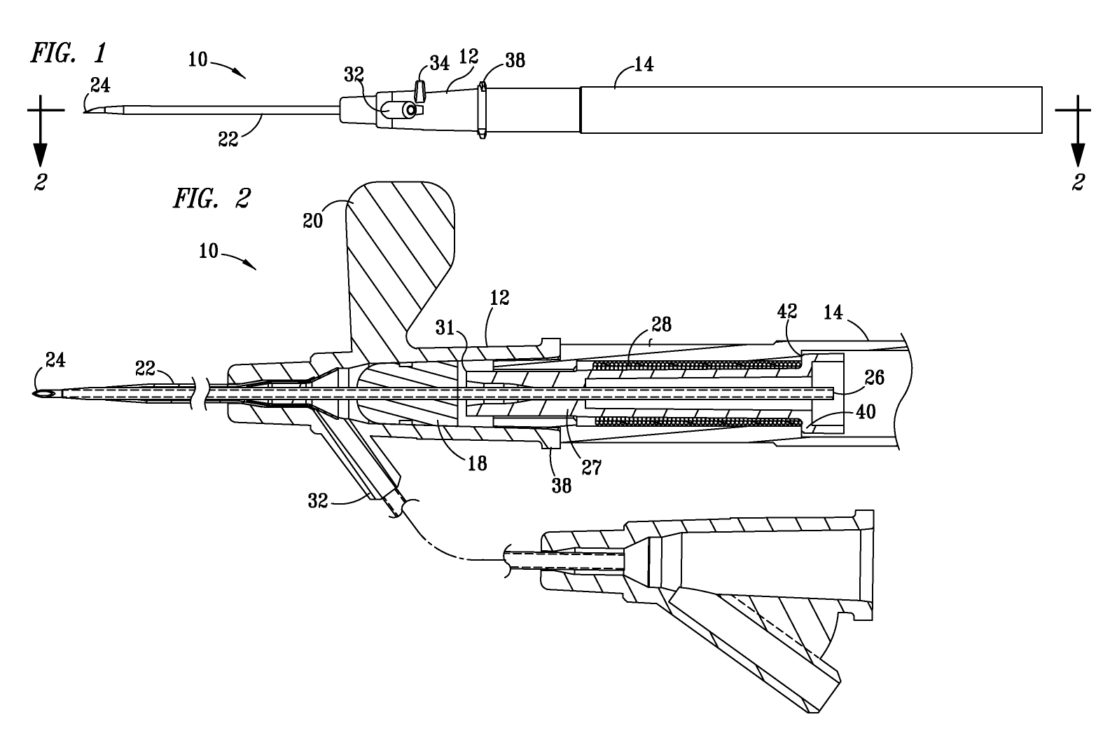

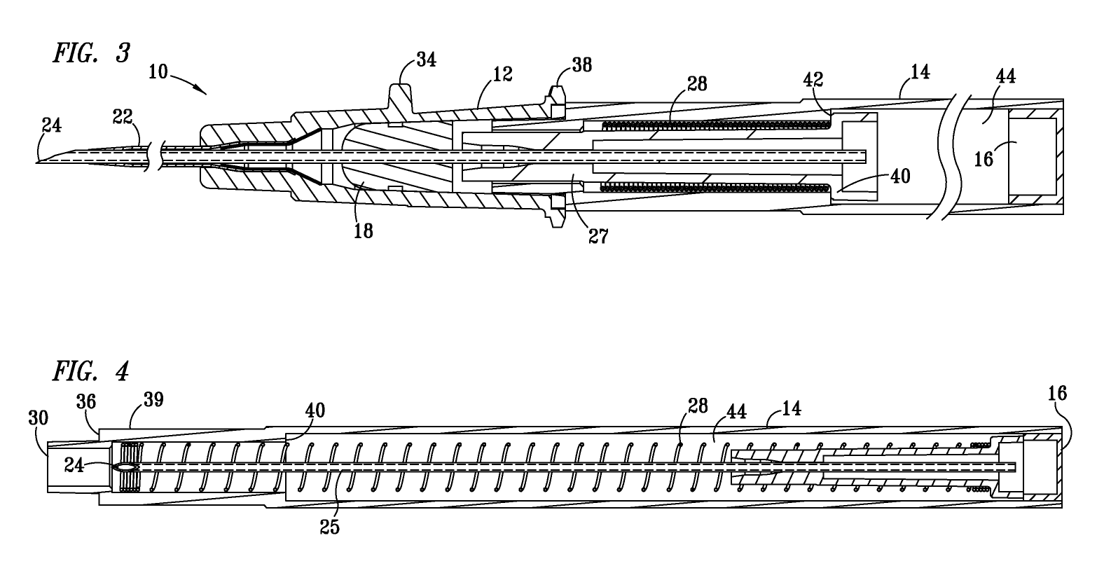

Referring to the embodiment of FIGS. 1-4, IV catheter introducer 10 comprises catheter hub 12, barrel 14, barrel end cap 16, catheter hub seal 18, transverse wing 20, catheter sheath 22, beveled needle end 24, needle 25, blunt needle end 26, needle holder 27, retraction spring 28, barrel front tip 30, needle holder front tip 31, lateral access port 32, finger stop 34, annular shoulder (barrel OD) 36, catheter hub flange 38, annular shoulder (barrel ID) 40, annular shoulder (needle holder) 42, and needle retraction chamber 44. All parts except for needle 25, retraction spring 28 and catheter hub seal 18 are preferably made of plastic suitable for use in such medical devices. Catheter hub seal 18 is preferably made of a polymeric material and more preferably made of an elastomeric material that is substantially fluid impermeable but has a centrally disposed longitudinal bore to receive needle 25.

During manufacture, catheter hub seal 18 is seated inside catheter hub 12 and front tip 30 of barrel 14 is inserted into catheter hub 12 through catheter hub flange 38. Needle 25 passes coaxially through catheter hub seal 18 and catheter sheath 22, with beveled tip end 24 extending slightly beyond the forwardly extending end of catheter sheath 22. The outside surface of nose 39 of barrel 14 can optionally frictionally engage the tapered inside wall of catheter hub 12, but this is not necessary, as the holding force exerted on needle 25 by catheter hub seal 18 is desirably greater than the biasing force exerted on needle holder 27 by retraction spring 28.

Prior to the insertion of cap 16 into the opening in the back of barrel 14, retraction spring is placed coaxially over needle holder 27 forwardly of annular shoulder 42 and needle holder 27 is inserted forwardly through retraction cavity 44 until retraction spring 28 is compressed. During assembly, retraction spring 28 can be held in compression by inserting a rod into barrel 14. Blunt end 26 of needle 25 desirably extends rearwardly through the longitudinally extending axial bore of needle holder 27 and needle 25 is secured in fixed relation to needle holder 27 by the use of cement or adhesives, or in any other similarly effective way known to those of ordinary skill in the art of making similar medical devices. When needle holder 27 is seated inside barrel 14, forwardly facing annular shoulder 42 of needle holder 27 is desirably disposed close to, or possibly abutting, rearwardly facing annular shoulder 40 inside barrel 14. When needle holder 27 is in this position, front end 31 of needle holder is desirably near but not touching catheter hub seal 18. Blunt end 26 of needle 25 desirably extends far enough past the bore through needle holder 27 that fluid is visible when beveled tip 24 is inserted into a vein, allowing needle retraction chamber 44 to serve also as a flash chamber. Alternatively, a notch (not shown) can be provided near beveled tip 24 of needle 25 to allow blood to show visibly in the catheter sheath to confirm to the clinician using the device that beveled needle tip 24 is inside a vein. Once the forwardly extending portion of catheter 22 has entered a vein, transverse wing 20 and catheter hub 12 are desirably taped in place on a patient prior to removing needle 25 from catheter hub 12.

When IV catheter introducer is configured as shown in FIGS. 1-3, needle holder 27 and needle 25 are maintained in their longitudinal positions relative to catheter hub 12 by a constrictive force exerted around the shaft of needle 25 by catheter hub seal 18. Optionally, such constrictive force can also be assisted by frictional engagement between the inside wall of catheter hub 12 and nose 39 of barrel 14.

After beveled tip 24 of needle 25 and the front end of catheter sheath 22 are inserted into a patient's vein, needle 25 is desirably withdrawn through catheter hub seal 18. This is done by exerting digital pressure against finger stop 34 and by manually pulling rearwardly on barrel 14 until front tip 24 of needle 25 is disposed rearwardly of catheter hub seal 18. As this step is performed by a clinician, barrel 14 also disengages from catheter hub 12 and the biasing force exerted rearwardly on annular shoulder 42 of needle holder 27 by retraction spring 28 drives needle holder 27 and spring 28 rearwardly into retraction cavity 44 of barrel 14. As needle 25 is withdrawn through catheter hub seal 18, the shaft of needle 25 is wiped as the bore of catheter hub seal 18 squeezes down around needle 25. After barrel 14 has been disengaged from catheter hub 12, it appears as depicted in FIG. 4, with retraction spring 28 relaxed and with the full length of needle 25 disposed inside retraction cavity 44.

Other alterations and modifications of the invention will likewise become apparent to those of ordinary skill in the art upon reading this specification in view of the accompanying drawings, and it is intended that the scope of the invention disclosed herein be limited only by the broadest interpretation of the appended claims to which the inventors are legally entitled.

We claim:

1. An IV catheter introducer comprising a catheter hub with a catheter sheath and a catheter hub seal disposed inside the catheter hub; a needle penetrating the catheter hub seal and projecting forwardly through the catheter sheath; a barrel; and a needle retraction mechanism that is activated by manual withdrawal of the needle from the catheter hub seal.

2. The IV catheter introducer of claim 1 wherein the catheter hub seal constricts to wipe the needle as the needle is manually withdrawn from the catheter hub seal.

3. The IV catheter introducer of claim 1 wherein the catheter hub seal constricts to block fluid flow rearwardly out of the catheter hub following manual withdrawal of the needle through the catheter hub seal.

4. An IV catheter introducer comprising a catheter hub and an automated needle retraction system having a needle that penetrates a catheter hub seal disposed inside the catheter hub, which catheter hub seal constricts to prevent a bodily fluid from flowing rearwardly out of the catheter hub after the needle is withdrawn past the seal.

5. A wholly passive medical catheter introducer having a needle and a catheter sheath, wherein incremental reinsertion of the needle into the catheter sheath is prevented by rearward, unidirectional movement of the needle rearwardly from the catheter sheath.

6. An IV catheter introducer comprising:
a catheter hub with a forwardly extending catheter sheath;
a catheter hub seal disposed inside the catheter hub;
a barrel having a front end insertable into an opening in the rear of the catheter hub behind the catheter hub seal, a selectively closed back end opposing the front end, and a retraction cavity disposed between the front end and the rear end;
a needle passing through an axial bore in the catheter hub seal and having a beveled tip projecting forwardly from the catheter sheath; and
a needle retraction assembly seated inside the barrel, the needle retraction assembly comprising a needle holder and a retraction spring, the needle holder and needle being maintained in position relative to the catheter hub and catheter sheath prior to retraction by a constrictive force exerted on the needle by the catheter hub seal.

7. The IV catheter introducer of claim 6 wherein withdrawal of the front tip of the barrel from the catheter hub and withdrawal of the needle from the catheter hub seal allows the retraction spring to force the needle holder and needle into the retraction cavity.

8. The IV catheter introducer of claim 6 wherein the catheter hub seal is made of a polymeric material.

9. The IV catheter introducer of claim 8 wherein the catheter hub seal is made of an elastomeric material.

10. The IV catheter introducer of claim 7, further comprising a lateral fluid access port disposed forwardly of the catheter hub seal and providing fluid communication with the interior of the catheter sheath after retraction of the needle.

11. The IV catheter introducer of claim 10 wherein the catheter hub seal constricts to close the axial bore and form a fluid seal following withdrawal of the needle from the bore.

12. The IV catheter introducer of claim 7 wherein the needle comprises a rear end that communicates a fluid flow rearwardly of the needle holder following insertion of the needle tip into a patient, thereby enabling the retraction cavity to also function as a flash chamber during insertion.

13. The IV catheter introducer of claim 7 wherein the barrel, catheter hub and needle holder are made of one or more moldable polymeric resins.

14. The IV catheter introducer of claim 6 wherein the needle holder is configured to orient the beveled needle tip upwardly relative to the catheter hub.
